(19) United States
(12) Patent Application Publication
(10) Pub. No.: US 2005/0215458 A1
(43) Pub. Date: Sep. 29, 2005

(54) Cleaning and sanitizing wipes

(75) Inventors: Robert Blaine Lalum, Antelope, CA (US); Asia Kogan, Citrus Heights, CA (US); Zhong Wei Xie, Folsom, CA (US); Yu Yao, Rocklin, CA (US); Gary Linn Robinson, Livermore, CA (US)

Correspondence Address: T. Ling Chwang;Suite 600, 2435 N. Central Expressway, Richardson, TX, 75080, US

(73) Assignee: Mionix Corporation, Rocklin, CA (US)

(21) Appl. No.: 10/950,960

(22) Filed: Sep. 27, 2004

Related U.S. Application Data
(60) Provisional application No. 60/508,080, filed on Oct. 2, 2003.

(51) Int. Cl. C11D 17/00

(52) U.S. Cl. 510/438

(57) ABSTRACT

A cleaning and sanitizing wipe, comprising a porous or absorbent sheet which has been infused with a cleaning and antimicrobial composition. The cleaning and antimicrobial composition contains a phenolic antimicrobial agent, an anionic surfactant, a hydric solvent, and water. Optionally, the cleaning and antimicrobial composition used in the wipes may contain a preservative, fragrance, and additional moisturizers. The pH of the cleaning and sanitizing wipes is preferably about 5.3 to about 6.5.

This application claims priority to U.S. Provisional Patent Application, Ser. No. 60/508,080, entitled “Sanitizing Wipes” filed on Oct. 2, 2003, the entire content of which is hereby incorporated by reference.

### BACKGROUND

This invention relates to surface-cleaning and antimicrobial sanitizing wipes comprising porous or absorbent sheets infused with a cleaning and antimicrobial composition containing a solubilized phenolic antimicrobial agent.

Topical cleaning and antimicrobial solutions, such as soaps and washes, are frequently used to clean and to minimize residual microbial presence and provide protection from future contamination on skin surfaces and other surfaces. These cleaning antimicrobial solutions typically contain one or more antimicrobial agents.

Antimicrobial agents are chemicals that kill or inhibit the growth of microbial organisms. Examples of antibacterial agents include are bisbiquanide, diphenyl compounds, benzyl alcohols, trihalocarbanilides, quaternary ammonium compounds, ethoxylated phenols, alcohols, cationic surfactants, and phenolic compounds. Phenolic antimicrobial agents kill microbial organisms through cell wall disruption and enzyme inactivation.

However, phenols have extremely low solubility in water. Typically, solubility of the phenolic antimicrobial agent is increased through the addition of surfactants. U.S. Pat. No. 6,451,748 to Taylor, et al. discloses an antibacterial composition containing a phenolic antimicrobial agent that is solubilized in a surfactant. Surfactants in water form micelles around the phenolic antimicrobial agent. These micelles allow dispersion of the agent in water.

One example of a phenolic antimicrobial agent is para-chloro-meta-xylenol (“PCMX”). PCMX is a desirable antimicrobial agent and is particularly effective against a wide variety of Gram positive and Gram negative bacteria. PCMX has a phenolic chemical structure and is related to compounds such as cresol, carbolic acid, and hexachloroprene. PCMX goes by a variety of other names, including chloroxylenol; 4-chloro-3,5xylenol; 4-chloro-3,5-dimethylphenol; 2-chloro-m-xylenol; 2-chloro-5-hydroxy-m-xylene; 2-chloro-5-hydroxy-m-xylene; 2-chloro-5-hydroxy-1,3-dimethylbenzene; 4-chlor-1-hydroxy-3,5-dimethyl benzene; and 3,5-dimethyl-4-chlorophenol. Antimicrobial formulations containing phenolic agents such as PCMX as disinfecting ingredients are known in the art and disclosed by Garabedian, et al., U.S. Pat. No.4,632,772; Corti, et al., U.S. Pat. No. 5,114,978; Kahn, et al., U.S. Pat. No. 5,439,681; Woodin, Jr., et al., U.S. Pat. No. 5,494,533; Fendler, et al., U.S. Pat. No. 5,635,462; Beerse, et al., U.S. Pat. No. 6,287,577; Childers, et al., U.S. Pat. No. 6,413,921; Sine, etal.,U.S. Pat. No.6,423,329; Stack, U.S. Pat. No.6,517,854; and Asmus,et al., U.S. Pat. No. 6,582,711.

Bacteria found on the skin can be divided into two groups: resident and transient bacteria. Resident bacteria are Gram positive bacteria which have been established as permanent microcolonies on the surface and outermost layers of the skin and play an important role in preventing the colonization of other, more harmful bacteria and fungi. Transient bacteria are not part of the normal resident flora of the skin, but can be deposited when airborne contaminated material lands on the skin or is brought into physical contact with it. Transient bacteria are typically Gram positive and Gram negative. Gram negative bacteria are generally distinguished from Gram positive by an additional protective cell membrane which generally results in their decreased susceptibility to topical antibacterial active agents.

Antimicrobial cleansing products have been marketed in a variety of forms for some time, including deodorant soaps, hard surface cleaners, and surgical disinfectants. These traditional rinse-off antimicrobial products have been formulated to provide bacteria reduction during washing. Some of these antimicrobial products, especially the hard surface cleaners and surgical disinfectants, utilize high levels of alcohol and/or harsh surfactants, which dry out and irritate skin tissues. Ideal personal cleaners should gently cleanse the skin, cause little or no irritation, not leave the skin overly dry after frequent use, and preferably provide a moisturizing benefit, while at the same time providing effective surface antimicrobial action and leaving residual antimicrobial agents to safeguard the surface without leaving an unpleasant film or odor.

Traditional antimicrobial compositions have also been developed for use in a washing process with water, which limits their availability when water is not available. By contrast, surface sanitizing and/or cleansing wipes have been used to wash the hands and face while traveling or in public when water is unavailable. For example, U.S. Pat. No.6,613,729 to Cole, et al. discloses the use of anionic surfactants and fatty acids on a wipe, but does not suggest the use of an antimicrobial active agent required to provide the improved residual effectiveness benefits. U.S. Pat. Nos. 6,488,943, 6,482,423, and 6,258,368 to Beerse, et al., disclose antimicrobial wipes using an antimicrobial agent, but which also utilize a proton-donating agent requiring a pH of 3.0 to 6.0 in the wipe solution.

### SUMMARY

This invention is directed to cleaning and sanitizing wipes containing a cleaning and antimicrobial composition with a pH of about 5.3 to about 6.5 and their methods of use. The cleaning and antimicrobial composition is infused into absorbent sheets and contains a solubilized phenolic antimicrobial agent such as p-chloro-m-xylenol (“PCMX”). The wipes may be used to clean and sanitize the skin or other surfaces.

The cleaning and sanitizing wipes are non-toxic, non-flammable, non-staining, and milder to the skin than many currently available products. The cleaning and antimicrobial composition used on the wipes combines a highly effective and fully solubilized phenolic antimicrobial agent with conditioners, emollients, and botanicals which moisturize the skin and allow the wipes to be used as a routine cleanser.

The cleaning and sanitizing wipe comprises a porous or absorbent sheet which has been infused or impregnated with a cleaning and antimicrobial composition. The cleaning and antimicrobial composition contains a mixture of several components. The bulk of the cleaning and antimicrobial composition is made up of deionized water. One active ingredient in the cleaning and antimicrobial composition is a phenolic antimicrobial agent, such as PCMX. Other ingredients include an anionic surfactant, such as ammonium lauryl sulfate, and a hydric solvent, such as hexylene glycol. Additional optional ingredients include a preservative such as phenoxyethanol, fragrance, moisturizers such as vitamin E succinate and aloe vera gel, and a pH adjuster.

The pH of the cleaning and antimicrobial composition used on the cleaning and sanitizing wipes is preferably between 5.3 and 6.5. PCMX has antimicrobial activity when it is present in a composition having a pH of between 4.0 and 9.0. The antimicrobial activity of PCMX is greater at the higher pH end of this range. Thus, the majority of PCMX-based soaps and disinfectants have a pH around 9.0. However, pH levels this high can be irritating to the skin. The pH of the cleaning and sanitizing wipes is closer to that which occurs naturally on the surface of the skin and is less irritating.

The cleaning and sanitizing wipes have an exceptionally high broad spectrum antibacterial efficacy, as measured by a rapid kill of bacteria. The cleaning and antimicrobial composition inhibits the growth of harmful microorganisms such as Salmonella choleraesuis, Pseudomonous aeruginosa, Staphylococcus aureus, and Escherichia coli. In particular, the cleaning and sanitizing wipes show a log reduction of at least about 3 to about 5.4 against Gram positive bacteria (e.g. S. aureus) and at least about 4 to about 5.8 against Gram negative bacteria (e.g., Escherichia coli) after about 30 seconds to about 3 minutes of contact.

The cleaning and sanitizing wipes are used without water, and the cleaning and antimicrobial composition remains on the skin or the surface after drying.

### DETAILED DESCRIPTION OF PREFERRED EMBODIMENTS

The current invention pertains to cleaning and sanitizing wipes comprising a porous or absorbent sheet which has been infused or impregnated with a cleaning and antimicrobial composition. The cleaning and antimicrobial composition used in the wipes contains a phenolic antimicrobial active agent and has a pH from about 5.3 to about 6.5. Other ingredients of the cleaning and antimicrobial composition include an anionic surfactant, a hydric solvent, and deionized water. Optional ingredients include a preservative, fragrance, and additional moisturizers.

A preferred embodiment of the cleaning and sanitizing wipes contains a phenolic antimicrobial active agent. The phenolic antimicrobial agent may be PCMX, 2,4,4′-trichloro-2′-hydroxy-diphenylether, benzylalkonium chloride, or 4-chloro-3,5-dimethylphenol. Preferably, the phenolic antimicrobial agent is PCMX. The phenolic antimicrobial agent may be present in the cleaning and antimicrobial composition from about 0. 1% to about 3.75% (by weight), preferably from at about 0.1% to about 0.6%, and more preferably from about 0.1% to about 0.3%.

In addition, a preferred embodiment of the cleaning and sanitizing wipes contains an anionic surfactant. Without wanting to be bound by theory, the anionic surfactant likely disrupts the lipids in the cell membrane of the bacteria and allows the antimicrobial agent to pass more easily through the weakened cell wall. The anionic surfactant also helps dissolve the phenolic antimicrobial agent. The anionic surfactant may be any anionic lathering surfactant, such as alkyl and alkyl ether sulfates, sulfated monoglycerides, sulfonated olefins, alkyl aryl sulfonates, primary or secondary alkane sulfonates, alkyl sulfosuccinates, acyl taurates, or acyl isethionates. Preferably, the anionic surfactant is a lauryl sulfate, such as ammonium lauryl sulfate, preferably at 28% concentration. The cleaning and antimicrobial composition may contain from about 0.95% to about 35.15% anionic surfactant, preferably from about 0.95% to about 5.7%, and more preferably from about 0.95% to about 2.85%.

A preferred embodiment of the cleaning and sanitizing wipes also includes a hydric solvent, such as a triol or diol. The hydric solvent may be propylene glycol, hexylene glycol, triethylene glycol, ethylene glycol, or diethylene glycol. Preferably, the hydric solvent is hexylene glycol, preferably at 98% concentration. The hydric solvent may be present from about 1% to about 8%, preferably from about 1% to about 5%, and more preferably from about 1% to about 2%.

A preferred embodiment of the cleaning and sanitizing wipes also contains water, preferably deionized or purified, in an amount to bring the mixture up to 100%.

A preferred embodiment of the cleaning and sanitizing wipes also optionally includes a preservative. The preservative may be phenoxyethanol, chlorphenesin, iodopropynyl butylcarbamate, benzoic acid, potassium sorbate, or sorbic acid. Preferably, the preservative is a phenoxyethanol-based preservative. The preservative may be present in the cleaning and antimicrobial composition from about 0.1% to about 1%, preferably from about 0.3% to about 0.7%, and more preferably from about 0.1% to about 0.5%.

An optional ingredient in additional preferred embodiments is a fragrance, which may be present from about 0.01% to about 0.5%, preferably about 0.01% to about 0.2%, and more preferably about 0.01% to about 0.05%. Any type of natural or artificial fragrance may be used. Other optional ingredients are additional moisturizers, such as vitamin E and aloe vera. Vitamin E may be present as vitamin E succinate, vitamin E acetate, or vitamin E (alpha tocopherol). Preferably, vitamin E succinate is used. The vitamin E may be present from about 0.005% to about 0.4%, preferably from about 0.005% to about 0.07%, and more preferably from about 0.005% to about 0.01%. Aloe vera may be present as a gel or extract, preferably a gel, from about 0.025% to about 1%, preferably from about 0.025% to about 0.1%, and more preferably from about 0.01% to about 0.03%.

An additional optional ingredient in a preferred embodiment is a pH adjuster. The pH adjuster may be acidic calcium sulfate (“ACS”), sodium hydroxide, potassium hydroxide, sulfuric acid, phosphoric acid, or any alpha hydroxy organic acid such as citric acid or lactic acid. The pH adjuster is preferably ACS, which may be present from about 0.01% to about 0.1%, preferably from about 0.01% to about 0.05%, and more preferably from about 0.01% to about 0.03%.

ACS may also be defined as an acidic, or low pH, solution of sparingly-soluble Group IIA-complexes (“AGIIS”) (See, U.S. patent application Ser. No. 09/500,473, “Acidic Solution of Sparingly-Soluble Group IIA Complex”; see also, U.S. Pat. No. 6,436,891, “Adduct Having An Acidic Solution of Sparingly-Soluble Group IIA Complexes”; the entire content of each of the two is hereby incorporated by reference). The term “complex,” as used herein, denotes a composition wherein individual constituents are associated. “Associated” means constituents are bound to one another either covalently or non-covalently, the latter as a result of hydrogen bonding or other inter-molecular forces. The constituents may be present in ionic, non-ionic, hydrated or other forms.

The acidic solution of sparingly-soluble Group IIA-complexes (“AGIIS”) can be prepared in several ways. Some of the methods involve the use of Group IA hydroxide but some of syntheses are devoid of the use of any added Group IA hydroxide, although it is possible that a small amount of Group IA metal may be present as “impurities.” The preferred way of manufacturing AGIIS (or ACS) is not to add Group IA hydroxide to the mixture. As the phrase implies, AGIIS is highly acidic, ionic, with a pH of below about 2.

The preferred method of preparing AGIIS (or ACS) involves mixing a mineral acid with a Group IIA hydroxide, or with a Group IIA salt of a dibasic acid, or with a mixture of the two Group IIA materials. In the mixing, a salt of Group IIA is also formed. Preferably, the starting Group IIA material or materials selected will give rise to, and form, the Group IIA salt or salts that are sparingly soluble in water. The preferred mineral acid is sulfuric acid, the prefered Group IIA hydroxide is calcium hydroxide, and the prefer Group IIA salt of a dibasic acid is calcium sulfate. Other examples of Group IIA salt include calcium oxide, calcium carbonate, and “calcium bicarbonate.”

AGIIS (or ACS) is preferably prepared by mixing calcium hydroxide with concentrated sulfuric acid, with or without an optional Group IIA salt of a dibasic acid (such as calcium sulfate) added to the sulfuric acid. For every mole of concentrated acid, such as sulfuric acid, the amount, in moles, of calcium hydroxide used is application specific and ranges from about 0.1 to about 1. The optional calcium sulfate can be added to the concentrated sulfuric acid prior to the introduction of calcium hydroxide into the blending mixture. The addition of calcium sulfate to the concentrated sulfuric acid appears to reduce the amount of calcium hydroxide needed for the preparation of AGIIS (or ACS). For every mole of concentrated acid, such as sulfuric acid, the amount, in moles, of calcium carbonate ranges from about 0.001 to about 0.2, depending on the amount of calcium hydroxide used. Other optional reactants include calcium carbonate and gaseous carbon dioxide being bubbled into the mixture. Regardless of the use of any optional reactants, the use of calcium hydroxide is desirable.

The following procedure may be used to make 1.2-1.5 N AGIIS (or ACS). An amount of 1055 ml (19.2 moles, after purity adjustment and taking into account the amount of acid neutralized by base) of concentrated sulfuric acid (FCC Grade, 95-98% purity) is slowly added with stirring, to 16.868 L of RO/DI water in each of reaction flasks a, b, c, e, and f. The amount of water is adjusted to allow for the volume of acid and the calcium hydroxide slurry. The mixture in each flask is mixed thoroughly. Each of the reaction flasks is chilled in an ice bath until the temperature of the mixture in the reaction flask is about 8-12° C. The mixture is continuously stirred at a rate of about 700 rpm.

Separately, a slurry is made by adding RO/DI water to 4 kg of calcium hydroxide (FCC Grace, 98% purity) making a final volume of 8 L. The mole ratio of calcium hydroxide to concentrated sulfuric acid is 0.45 to 1. The slurry is a 50% (WNV) mixture of calcium hydroxide in water. The slurry is mixed well with a high-shear-force mixer until the slurry appears uniform. The slurry is then chilled to about 8-12° C. in an ice bath and continuously stirred at about 700 rpm.

To each of the reaction flasks is added 150 ml of the calcium hydroxide slurry every 20 minutes until 1.276 L (i.e. 638 g dry weight, 8.61 moles, of calcium hydroxide) of the slurry has been added to each reaction vessel. The addition is again accompanied by mixing at about 700 rpm. After the completion of the addition of the calcium hydroxide to the reaction mixture in each reaction vessel, the mixture is filtered through a 5-micron filter. The filtrate is allowed to sit for 12 hours, then the clear solution is decanted to discard any precipitate formed. The resulting product is AGIIS (or ACS) having an acid normality of 1.2-1.5.

The final pH of the cleaning and antimicrobial composition used in the cleaning and sanitizing wipes preferably ranges from about 5.3 to about 6.5.

The cleaning and antimicrobial composition is added to one or both sides of an absorbent sheet or wipe. The wipe may be formed from any woven or nonwoven fiber, fiber mixture or foam of sufficient wet strength and absorbency to hold an effective amount of the cleaning and antimicrobial composition. For an exemplary use as a consumer cleaning and sanitizing wipe, the wipe should preferably measure about 6 to 8 inches on each side. About 3 g to about 8 g of the cleaning and antimicrobial composition is added to each wipe, preferably about 3 g to about 6 g, and more preferably about 4 g to about 5 g. The wipes may be of any desired thickness or dimension which allows absorption of the preferred amount of cleaning and antimicrobial composition.

A preferred embodiment of the cleaning and sanitizing wipes uses an absorbent sheet or wipe which has an ideal gravimetric basis weight of about 2.0 oz/yd² and an ideal tensile strength of about 20 to 28 pounds. One particular example of an absorbent sheet or wipe which may be used in the cleaning and sanitizing wipes (Sontara® Spunlaced Fabric Style S-P005, DuPont, Wilmington, Del.) may be obtained as a roll and cut to the desired size. These wipes have an individual gravimetric basis weight ranging from about 1.67 to 2.33 oz/yd², a roll average gravimetric basis weight ranging from about 1.82 to 2.18 oz/yd², an individual tensile strength of about 18.05 lbs., and a roll average tensile strength of about 22.98 lbs.

To produce the cleaning and antimicrobial composition to be used on the cleaning and sanitizing wipes, the deionized water is first measured out into a container. The preservative and hydric solvent are then added. The solution is mixed and heated to about 40-45° C. The phenolic antimicrobial agent is then sprinkled in, and the solution is mixed for at least 20-30 minutes. Mixing is then slowed to eliminate the vortex, and the anionic surfactant is added slowly. This solution is mixed at a moderate speed to avoid foaming for about 2 hours to dissolve the phenolic antimicrobial agent. An optional pH adjuster such as ACS may be added at this point. The solution is then cooled to about 35-38° C. while mixing. Separately, an additional moisturizer, such as vitamin E, can be dissolved in an optional fragrance. This mixture is then slowly added to the first solution and mixed constantly at a temperature of about 35-38° C. until the solution is uniform. Another additional moisturizer, such as aloe vera gel, may then be added and mixed thoroughly at an appropriate speed until the solution is uniform and clear. The pH adjuster, additional moisturizers, and fragrance can be added in a different order than described herein, depending on the preference of the operator and the desired final composition.

In a preferred embodiment, the cleaning and sanitizing wipes are prepared by wetting the absorbent sheets with the cleaning and antimicrobial composition. The absorbent sheets may be infused with the cleaning and antimicrobial composition by any suitable means, such as spraying or dipping. In one preferred method, rolls of absorbent material are passed over a wetting tube which dispenses the cleaning and antimicrobial composition through a series of small holes onto the material. The wetting tube is connected to a pump that meters the appropriate amount of liquid dispensed onto the absorbent material as it passes over the wetting tube. The wetting process may be controlled by weighting the resulting wipe stacks and adjusting the dispensing or metering pump until the appropriate total weight of the stack is obtained. A machine (Clipper™ Series RX-300C, Paper Converting Machine Company, Green Bay, Wis.) may be used to wet, cut, fold, and stack the wetted wipes into the desired size and number. After the wetting process is complete, the stacks of wipes may be placed in the interior of a container, such as a plastic tub. The container should provide a substantially hermetically sealed environment to minimize the escape of any of the cleaning and antimicrobial composition.

The cleaning and sanitizing wipes are capable of producing a log reduction of Gram positive bacteria of about 3 to about 5.4 and of Gram negative bacteria of about 4 to about 5.8 after about 30 seconds to 3 minutes of contact, as measured against Staphylococcus aureus, Salmonella choleraesuis, Pseudomonas aeroginosa, and Escherichia coli. The cleaning and sanitizing wipes may preferably be used on the skin or other surfaces.

### EXAMPLE 1

### Preparation and Experimental Procedures

Preparation of Cleaning and Antimicrobial Composition and Sanitizing Wipes

To prepare the cleaning and antimicrobial composition and the cleaning and sanitizing wipes, the following procedure was used. The specific amounts of each component vary according to the desired final composition.

A predetermined amount of deionized water was first measured into a container. Then, phenoxyethanol (Phenoxytol, Clariant, Muttenz, Switzerland) and hexylene glycol (98%, Spectrum, Gardena, Calif.) were added, according to the predetermined amounts. The composition was mixed at a moderate speed and heated to about 40-45° C. Then, PCMX (Nipacide® MX, Clariant) was sprinkled in and the composition was mixed for 20-30 minutes.

After eliminating the vortex, ammonium lauryl sulfate (28%, Stepanol® AM-V, Stepan, Northfield, Ill.) was added slowly. The composition was then mixed at a moderate constant speed, to avoid foaming, for about two hours, until the PCMX was dissolved. The uniformity of the solution and the absence of undissolved solid material was verified.

Next, ACS (0.15N, Mionix, Rocklin, Calif.) was added and the composition was mixed for 15-20 minutes and then cooled to 35-38° C. In a separate container, vitamin E succinate (Spectrum Chemical, Gardena, Calif.) was dissolved in the fragrance (“Tropical Kiwi,” Arylessence, Inc., Marietta, Ga.) and mixed until uniform. The vitamin E and fragrance mixture was then added to the composition slowly, with constant mixing and maintaining a temperature of about 35-38° C. The composition was mixed until uniform. Next, the aloe vera gel (Aloe-Active Gel-D, Aloecorp, Broomfield, Colo.) was added and the composition was mixed thoroughly at an appropriate speed until uniform and clear. The pH of the composition was checked and determined to be within the range of about 5.3 to about 6.5.

To produce the cleaning and sanitizing wipes, rolls of absorbent material (Sontara® Spunlaced Fabric Style S-P005, DuPont, Wilmington, Del.) were run through a machine (Clipper™ Series RX-300C, Paper Converting Machine Company, Green Bay, Wis.). The machine passed the absorbent material over a wetting tube, which dispensed the desired amount of the cleaning and antimicrobial composition onto the material. The machine then cut, folded, and stacked the cleaning and sanitizing wipes in the appropriate dimensions and number.

1.2 Wipes Time Kill Study

To determine the antimicrobial efficacy of samples of the cleaning and sanitizing wipes, a time kill study was performed according to the following procedure.

100 μl of the each tested bacterial culture, including (1) Salmonella choleraesuis ATCC #6539, overnight culture, (2) Pseudomonous aeruginosa ATCC #9027, overnight culture, (3) Staphylococcus aureus ATCC #6538, overnight culture, and (4) Escherichia coli O157:H7 ATCC #43894, overnight culture (ATCC, Manassas, Va.), was inoculated into petri dishes (divided into control and treatment groups) by striking with a pipette. The inoculated dishes were left open and dried inside a hood for 1 hour. Control samples of wipes contained either deionized water or phosphate buffer (pH 7.38). Each group of inoculated dishes was wiped with one of the sample wipe treatments in 25 circles, about 1 circle per second. After 30 seconds, 5 mL recovery broth (Letheen broth) was added to each individual petri dish, which was then swirled 15 times. A 10-fold dilution with phosphate buffer (pH 7.38) was then done for each dish. Different dilution levels of the samples (10⁰, for recovery broth, up to 10⁻⁴) were then plated onto TSA plates. The plates were stored upside down in a 37° C. incubator for about 40-48 hours. The colonies were then counted and the CFU were calculated. A viability count on an untreated petri dish may also be performed. The log reduction of each treatment was also calculated.

1.3 Cleaning and Antimicrobial Composition Time Kill Study

To determine the antimicrobial efficacy of samples of the cleaning and antimicrobial composition to be used on the cleaning and sanitizing wipes, a time kill study was performed according to the following procedure.

200 μl of each tested bacterial culture, including (1) Salmonella choleraesuis ATCC #6539, overnight culture, (2) Pseudomonous aeruginosa ATCC #9027, overnight culture, (3) Staphylococcus aureus ATCC #6538, overnight culture, and (4) Escherichia coli O157:H7 ATCC #43894, overnight culture (ATCC, Manassas, Va.), was added to separate 10 mL test tubes containing 4 mL of each cleaning and antimicrobial composition sample. Control samples of the compositions contained either deionized water or phosphate buffer (pH 7.38). The test tubes were mixed well immediately without allowing the pipette to touch the wall of the tubes. After 30 seconds, 0.5 mL of each test tube mixture was transferred to another test tube containing 5 mL of recovery broth (Letheen broth) and mixed well. A 10-fold dilution with phosphate buffer (pH 7.38) was then done for each tube. Different dilution levels of the samples (10⁰, for recovery broth, up to 10⁻⁴) were then plated onto TSA plates. The plates were stored upside down in a 37° C. incubator for about 40-48 hours. The colonies were then counted and the CFU were calculated. The log reduction of each sample composition was also calculated.

### EXAMPLE 2

### Components of Cleaning and Antimicrobial Composition

To determine the appropriate components for use in the cleaning and antimicrobial composition, three samples (Samples C1, C2, and C3) of the cleaning and antimicrobial composition to be used on the cleaning and sanitizing wipes were prepared in accordance with the procedure described in Example 1. Varying amounts of hexylene glycol and ACS were used. Table 2 below shows the components of Samples C1-C3.

TABLE 2
Cleaning and Antimicrobial Composition
Samples C1-C3 Components

| | W/W % | | |
|---|---|---|---|
| Component | Sample C1 | Sample C2 | Sample C3 |
| Deionized water | Up to 100% | Up to 100% | Up to 100% |
| Ammonium lauryl sulfate, | 2.85 | 2.84 | 2.85 |
| 28% | | | |
| Hexylene glycol | — | — | 5.0 |
| ACS, 0.15 N | — | 0.38 | — |
| pH | 6.8 | 5.3 | 6.8 |

Three days after the samples were prepared, precipitation of PCMX was observed in Samples C1 and C2. Thus, the results indicate that hexylene glycol is required to dissolve PCMX.

### EXAMPLE 3

### Cleaning and Sanitizing Wipes Preparation

An example (Sample W1) of the cleaning and sanitizing wipes was prepared in accordance with the procedure described in Example 1, but with modifications after the addition of ACS. After the ACS was added, 1 g of aloe vera gel was then added and the composition was mixed thoroughly at an appropriate speed until it was uniform and clear. 1 g of fragrance was added next and the composition was again mixed until uniform and clear. Vitamin E succinate was not added. The final pH of the mixture used on Sample W1 was then checked and determined to be 6.0. Table 3 below shows the components of Sample W1.

TABLE 3
Wipes Sample W1 Components
Sample W1

| Component | In 2000 g | W/W % |
|---|---|---|
| Deionized water | To 2000 g | Up to 100% |
| Phenoxyethanol | 10.0 | 0.5 |
| Hexylene glycol, 98% | 44.0 | 2.2 |
| PCMX | 5.0 | 0.25 |
| Ammonium lauryl sulfate | 33.0 | 1.65 |
| ACS 0.15 N | 0.8 | 0.04 |
| Fragrance | 1.0 | 0.05 |
| Aloe vera gel | 1.0 | 0.05 |

In order to produce the cleaning and antimicrobial composition to be used on a second example of the cleaning and sanitizing wipes (Sample W2), 1000 g of the cleaning and antimicrobial composition prepared for use in Sample W1 was combined with 0.25 g of ACS and mixed for 20 minutes. The final pH of the composition used on Sample W2 was determined to be 5.5.

To prepare the wipes of Sample W1 and Sample W2, 4 g of the respective cleaning and antimicrobial compositions was added to each 6.5 ×7 inch absorbent sheet (Sontara® Spunlaced Fabric Style S-P005, DuPont).

### EXAMPLE 4

### Cleaning and Sanitizing Wipes Comparative Study

The antimicrobial activity of the cleaning and sanitizing wipes of Sample W1 and Sample W2 was tested and compared to the antimicrobial activity of other commercially available cleaning and sanitizing wipes using the Wipe Time Kill Study described in Example 1 and the Gram positive bacteria Staphylococcus aureus.

Clorox™ wipes (Clorox, Oakland, Calif.) were used for comparison. The 7×8 inch Clorox™ wipes contain two active antimicrobial agents: dimethyl benzyl ammonium chloride (0.145%) and dimethyl ethylbenzyl ammonium chloride (0.145%). A control wipe was also prepared by adding 4 g deionized water to a 6.5×7 inch wipe. The results are shown in Table 4 below.

TABLE 4
Wipe Samples W1, W2, and Comparative Time Kill Data

| | S. Aureus | S. Aureus |
|---|---|---|
| Sample | (CFU/mL) | Log Reduction |
| Sample W1 | 6.0 × 10¹ | 2.89 |
| Sample W2 | 5.47 × 10¹ | 2.93 |
| Clorox^{TM} Wipes | 9.47 × 10¹ | 2.69 |
| Control | 4.67 × 10⁴ | — |

The results indicate that these examples (Samples W1and W2) of cleaning and sanitizing wipes are capable of producing a log reduction of at least two against Gram positive bacteria. Samples W1 and W2 also showed a higher log reduction than the commercially available Clorox™ wipes.

### EXAMPLE 5

### Cleaning and Antimicrobial Composition Study

Three additional examples (Samples C4, C5, and C6) of the cleaning and antimicrobial composition to be used on the cleaning and sanitizing wipes were prepared. Sample C4 contained the pH adjuster ACS and the active ingredient PCMX. Sample C5 lacked ACS. Sample C6 lacked ACS and PCMX. The procedure described in Example 1 was used to prepare the samples. The pH of Samples C4, C5, and C6 was determined to be 6.2, 6.4, and 6.0 respectively. Table 5-1 below shows the components of the samples.

TABLE 5-1
Cleaning and Antimicrobial Composition Samples C4-C6 Components

| | Sample C4 | | Sample C5 | | Sample C6 | |
|---|---|---|---|---|---|---|
| Component | In 2000 g | W/W % | In 2000 g | W/W % | In 2000 g | W/W % |
| Deionized water | Up to | To 100% | Up to | To 100% | Up to | To 100% |
| | 2000 g | | 2000 g | | 2000 g | |
| Phenoxyethanol | 10.0 | 0.5 | 10.0 | 0.5 | 10.0 | 0.5 |
| Hexylene glycol, 98% | 40.0 | 2.0 | 40.0 | 2.0 | 40.0 | 2.0 |
| PCMX | 6.0 | 0.3 | 6.0 | 0.3 | — | — |
| Ammonium lauryl sulfate, 28% | 57.0 | 2.85 | 57.0 | 2.85 | 57.0 | 2.85 |
| ACS, 0.15 N | 0.9 | 0.045 | — | — | — | — |
| Fragrance | 1.0 | 0.05 | 1.0 | 0.05 | 1.0 | 0.05 |
| Vitamin E Succinate | 0.2 | 0.01 | 0.2 | 0.01 | 0.2 | 0.01 |
| Aloe vera gel | 1.0 | 0.05 | 1.0 | 0.05 | 1.0 | 0.05 |

Samples C4, C5, and C6 were tested for antimicrobial activity using the Solution Time Kill Study described in Example 1, with all four listed bacterial cultures. The plate count data (CFU) are shown in Table 5-2 below.

TABLE 5-2
Cleaning and Antimicrobial Composition
Samples C4-C6 Plate Count Data

| | S. Choleraesuis | P. Aeruginosa | S. Aureus | E. Coli |
|---|---|---|---|---|
| Sample | (CFU/mL) | (CFU/mL) | (CFU/mL) | (CFU/mL) |
| C4 | <6.67 | <6.67 | 2.03 × 10⁵ | <6.67 |
| C5 | <6.67 | <6.67 | 6.27 × 10⁵ | <6.67 |
| C6 | 6.60 × 10⁵ | 1.29 × 10⁷ | 8.07 × 10⁶ | 1.51 × 10⁷ |
| Control | 2.27 × 10⁶ | 1.74 × 10⁷ | 4.39 × 10⁶ | 1.24 × 10⁷ |

The plate-count data (CFU) were converted to log scale reduction, as shown below in Table 5-3.

TABLE 5-3
Cleaning and Antimicrobial Composition
Samples C4-C6 Log Scale Reduction

| | S. Choleraesuis | P. Aeruginosa | S. Aureus | E. Coli |
|---|---|---|---|---|
| Sample | (Log) | (Log) | (Log) | (Log) |
| C4 | >5.54 | >6.42 | 1.33 | >6.27 |
| C5 | >5.54 | >6.42 | 0.6 | >6.27 |
| C6 | 0.55 | 0.13 | 0 | 0 |

The results indicate that the active antimicrobial ingredient (PCMX) is required for the cleaning and antimicrobial composition to work effectively, because Sample C6 showed no antimicrobial activity. The results also indicate that these samples of the cleaning and antimicrobial composition to be used on the cleaning and sanitizing wipes have high antimicrobial efficacy against Gram negative organisms (S. Choleraesuis, P. Aeruginosa, and E. Coli), but relatively little antimicrobial efficacy against Gram positive organisms (S. Aureus).

### EXAMPLE 6

### Cleaning and Sanitizing Wipes Study

Another sample of the cleaning and sanitizing wipes was created by modifying the cleaning and antimicrobial composition of Sample C5, used in Example 5 above. First, wipes (6×8 inch) were wetted with 4 g of Sample C5, to produce Wipes Sample W3. Then, a group of these wipes (Sample W4) were further wetted with an additional 0.3% PCMX dissolved in 1.0% hexylene glycol, in a total amount of 2 g. A control group was also prepared by wetting a group of wipes with 5 g of sterile deionized water.

Wipes Sample W3 and Sample W4 were tested for antimicrobial efficacy using the Wipes Time Kill Study described in Example 1 and all four listed bacterial cultures. Six petri dishes were inoculated with each bacterium, then divided into three groups of two. Each group of two petri dishes was wiped with one of the three wipe treatments. The plate count data, along with the initial inoculation amounts, are shown in Table 6-1 below.

TABLE 6-1
Wipes Samples W3 and W4 Plate Count Data

| | E. Coli | P. Aeruginosa | S. Choleraesuis | S. Aureus |
|---|---|---|---|---|
| Sample | (CFU/mL) | (CFU/mL) | (CFU/mL) | (CFU/mL) |
| Inocu- | 2.72 × 10⁸ | 2.67 × 10⁸ | 5.73 × 10⁷ | 1.31 × 10⁸ |
| lation | | | | |
| W3 | 9.00 × 10² | 1.67 × 10¹ | <1.67 × 10¹ | <1.67 × 10¹ |
| W4 | 1.67 × 10¹ | <1.67 × 10¹ | <1.67 × 10¹ | <1.67 × 10¹ |
| Control | 3.02 × 10⁵ | 2.59 × 10⁵ | 1.81 × 10⁴ | 5.60 × 10³ |

The plate count data were used to calculate the log scale reduction, as shown in Table 6-2 below.

TABLE 6-2
Wipes Samples W3 and W4 Log Scale Reduction

| | E. Coli | P. Aeruginosa | S. Choleraesuis | S. Aureus |
|---|---|---|---|---|
| Sample | (Log) | (Log) | (Log) | (Log) |
| W3 | 2.53 | 4.19 | 3.04 | 1.68 |
| W4 | 4.26 | >4.19 | >3.04 | 2.53 |

The results indicate that Sample W4 possesses an improved antimicrobial efficacy against Gram positive microorganisms such as S. Aureus, when compared to Sample W3.

### EXAMPLE 7

### Cleaning and Sanitizing Wipes Study Using Different Time Kill Test Method

An additional example (Sample W5) of the cleaning and sanitizing wipes was prepared according to the procedure described in Example 1 above. A control sample of wipes containing only 4 g deionized water was also prepared. The components of Sample W5 are shown in Table 7-1 below.

TABLE 7-1
Wipes Sample W5 Components

| | Sample W5 |
|---|---|
| Component | W/W % |
| Deionized water | To 100% |
| Phenoxyethanol | 0.5 |
| Hexylene glycol, 98% | 2.0 |
| PCMX | 0.3 |
| Ammonium lauryl sulfate, 28% | 2.85 |
| ACS, 0.15 N | 0.045 |
| Fragrance | 0.05 |
| Vitamin E Succinate | 0.01 |
| Aloe vera gel | 0.05 |

A different time kill study, to determine the antimicrobial efficacy of the wipes when used in a more realistic manner, was performed in two parts using Sample W5. In the first part, the study utilized: (1) Escherichia coli O157:H7 ATCC #43894, overnight culture in MDI E.C. medium and (2) Staphylococcus aureus ATCC #6538, overnight culture in Micrococcus medium. In the second part, the study utilized: (1) Pseudomonous aeruginosa ATCC #9027, overnight culture in nutrient medium and (2) Salmonella choleraesuis ATCC #6539, overnight culture in BHI broth.

The procedure performed for each part of the time kill study was the same. The strains of bacteria were cultured overnight at 37° C. in a water bath shaker. The bacteria were then harvested by centrifuging at 2500 rpm for 8 minutes. 90% of the supernatant was removed carefully by pipette and discarded. The pellets of bacteria left in the centrifuge tubes were then mixed by vortex and combined. The strain suspension for each bacterium equaled 2.2 mL. 116 μl of bovine serum (Lot #57H9307, Sigma, St. Louis, Mo.) was added into each tube containing 2.2 mL of the strain suspension. All tubes were then mixed well by vortex to produce a ready-to-use strain suspension with a 5% serum concentration. 100 μl of each ready-to-use strain suspension was inoculated into different petri dishes (21 dishes for each bacterium). The inoculated dishes were left open and dried inside a hood for about 30 minutes.

Each inoculated dish was then treated with a wipe, which was rubbed on the dish in ten circles, at about one circle per second. The same wipe was used to cover the contaminated surface for a total of 30 seconds, starting at the time of the first circle. After 30 seconds, the wipe was immediately removed. The dish was then covered and allowed to rest for 2 minutes and 30 seconds, allowing the bacteria remaining on the plate to be exposed to the wipe's cleaning and antimicrobial composition residue. Thus, the total kill time for each dish beginning with the first wipe circle was 3 minutes. After this time, 5 mL of recovery broth (Letheen broth) was added to each dish and swirled 15 times.

For the simultaneous viability comparison sample, 5 mL of recovery broth was added to an un-wiped dish from each bacterium and swirled to wash the bacteria until the striking line was no longer visible. The inoculated dishes were therefore treated as shown below in Table 7-2.

TABLE 7-2
Wipes Sample W5 Study Treatments

| | Number of Inoculated Dishes Tested | | | |
|---|---|---|---|---|
| Sample | E. Coli | S. Aureus | P. Aeruginosa | S. Choleraesuis |
| Viability | 1 | 1 | 1 | 1 |
| W5 | 10 | 10 | 10 | 10 |
| Control | 10 | 10 | 10 | 10 |

A ten-fold dilution was then carried out for each dish with phosphate buffer (pH 7.38). Different dilutions were then plated onto TSA plates. The recovery broth was counted as the 10⁰ dilution level. All plates were then incubated in a 37° C. incubator for about 40-48 hours. The colonies were then counted and the CFU calculated. The results, along with the inoculation amounts and viability comparison samples, are shown in Table 7-3 below.

TABLE 7-3
Wipes Sample W5 Plate Count Data

| | E. Coli | S. Aureus | P. Aeruginosa | S. Choleraesuis |
|---|---|---|---|---|
| Sample | (CFU/mL) | (CFU/mL) | (CFU/mL) | (CFU/mL) |
| Inoculation | 2.62 × 10⁹ | 6.27 × 10⁹ | 3.10 × 10⁹ | 8.53 × 10⁸ |
| Viability | 3.70 × 10⁸ | 1.03 × 10⁹ | 5.10 × 10⁸ | 2.53 × 10⁷ |
| W5 | 3.33 | 1.33 × 10¹ | 1.83 × 10² | <3.33 |
| Control | 2.24 × 10⁶ | 3.62 × 10⁶ | 2.67 × 10⁶ | 8.46 × 10⁴ |

The plate count data were used to calculate the log values and log scale reduction, as shown in Table 7-4 below.

TABLE 7-4
Wipes Sample W5 Log Value and Log Scale Reduction

| | E. Coli | S. Aureus | P. Aeruginosa | S. Choleraesuis |
|---|---|---|---|---|
| Sample | (Log) | (Log) | (Log) | (Log) |
| Log Values | | | | |
| Inoculation | 9.42 | 9.80 | 9.49 | 8.93 |
| Viability | 8.57 | 9.01 | 8.71 | 7.40 |
| W5 | 0.52 | 1.12 | 2.26 | <0.52 |
| Control | 6.35 | 6.56 | 6.43 | 4.93 |
| Log Scale | | | | |
| Reduction | | | | |
| W5 | 5.83 | 5.43 | 4.16 | >4.40 |

The results indicate that the cleaning and sanitizing wipes can achieve a log reduction of about 4 to about 6 against Gram negative and Gram positive bacteria.

### References Cited

The following U.S. Patent documents are hereby incorporated by reference.

### U.S. Patents

U.S. Pat. No. 4,632,772 to Garabedian, et al.
U.S. Pat. No. 5,224,978 to Corti, et al.
U.S. Pat. No. 5,439,681 to Kahn, et al.
U.S. Pat. No. 5,494,533 to Woodin, Jr., et al.
U.S. Pat. No. 5,635,462 to Fendler, et al.
U.S. Pat. No. 6,258,368 to Beerse, et al.
U.S. Pat. No. 6,287,577 to Beerse, et al.
U.S. Pat. No. 6,413,921 to Childers, et al.
U.S. Pat. No. 6,423,329 to Sine, et al.
U.S. Pat. No. 6,451,748 to Taylor, et al.
U.S. Pat. No. 6,482,423 to Beerse, et al.
U.S. Pat. No. 6,488,943 to Beerse, et al.
U.S. Pat. No. 6,517,854 to Stack, et al.
U.S. Pat. No. 6,582,711 to Asmus, et al.
U.S. Pat. No. 6,613,729 to Cole, et al.

1. A cleaning and sanitizing wipe comprising a porous or absorbent sheet infused with a cleaning and antimicrobial composition, wherein the cleaning and antimicrobial composition comprises a phenolic antimicrobial agent, an anionic surfactant, a hydric solvent, and water.

2. The cleaning and sanitizing wipe of claim 1, wherein the cleaning and antimicrobial composition further comprises a preservative, fragrance, additional moisturizer, and a pH adjuster.

3. The cleaning and sanitizing wipe of claim 3, wherein the phenolic antimicrobial agent is chloroxylenol (“PCMX”), the anionic surfactant is ammonium lauryl sulfate, the hydric solvent is hexylene glycol, the preservative is phenoxyethanol, and the pH adjuster is acidic calcium sulfate (“ACS”).

4. A cleaning and sanitizing wipe comprising a porous or absorbent sheet infused with a cleaning and antimicrobial composition, wherein the cleaning and antimicrobial composition comprises, by weight:
(a) From about 0.1% to about 3.75% of a phenolic antimicrobial agent;
(b) From about 0.95% to about 35.15% of an anionic surfactant;
(c) From about 1% to about 8% of a hydric solvent; and
(d) Remainder water.

5. The cleaning and sanitizing wipe of claim 4, wherein the phenolic antimicrobial agent is chloroxylenol (“PCMX”), 2,4,4′-trichloro-2′-hydroxy-diphenylether, benzylalkonium chloride, or 4-chloro-3,5-dimethylphenol.

6. The cleaning and sanitizing wipe of claim 4, wherein the anionic surfactant is an alkyl sulfate, an alkyl ether sulfate, a sulfated monoglyceride, a sulfonated olefin, an alkyl aryl sulfonate, a primary alkane sulfonate, a secondary alkane sulfonate, an alkyl sulfosuccinate, an acyl taurate, or an acyl isethionate.

7. The cleaning and sanitizing wipe of claim 4, wherein the hydric solvent is propylene glycol, hexylene glycol, triethylene glycol, ethylene glycol, or diethylene glycol.

8. The cleaning and sanitizing wipe of claim 4, wherein the cleaning and antimicrobial composition comprises, by weight:
(a) From about 0.1% to about 0.6% of a phenolic antimicrobial agent;
(b) From about 0.95% to about 5.7% of an anionic surfactant;
(c) From about 1% to about 5% of a hydric solvent; and
(d) Remainder water.

9. The cleaning and sanitizing wipe of claim 4, wherein the cleaning and antimicrobial composition comprises, by weight:
(a) From about 0.1% to about 0.3% of a phenolic antimicrobial agent;
(b) From about 0.95% to about 2.85% of an anionic surfactant;
(c) From about 1% to about 2% of a hydric solvent; and
(d) Remainder water.

10. The cleaning and sanitizing wipe of claim 4, wherein the cleaning and antimicrobial composition further comprises from about 0.1% to about 1% of a preservative.

11. The cleaning and sanitizing wipe of claim 10, wherein the preservative is phenoxyethanol, chlorphenesin, iodopropynyl, butylcarbamate, benzoic acid, potassium sorbate, or sorbic acid.

12. The cleaning and sanitizing wipe of claim 4, wherein the cleaning and antimicrobial composition further comprises from about 0.01% to about 0.05% of a fragrance.

13. The cleaning and sanitizing wipe of claim 4, wherein the cleaning and antimicrobial composition further comprises an additional moisturizer.

14. The cleaning and sanitizing wipe of claim 13, wherein the additional moisturizer is vitamin E, vitamin E succinate, vitamin E acetate, aloe vera, a polyol, or a mixture thereof.

15. The cleaning and sanitizing wipe of claim 14, wherein the additional moisturizer comprises from about 0.005% to about 0.4% of vitamin E succinate.

16. The cleaning and sanitizing wipe of claim 14, wherein the additional moisturizer comprises from about 0.025% to about 1% of aloe vera.

17. The cleaning and sanitizing wipe of claim 14, wherein the additional moisturizer comprises polyol, and wherein the polyol is sorbitol, mannitol, maltitol, isomalt, xylitol, erythritol, or a mixture thereof.

18. The cleaning and sanitizing wipe of claim 4, wherein the cleaning and antimicrobial composition further comprises from about 0.01% to about 0.1% of a pH adjuster.

19. The cleaning and sanitizing wipe of claim 18, wherein the pH adjuster is acidic calcium sulfate (“ACS”), sodium hydroxide, potassium hydroxide, citric acid, lactic acid, sulfuric acid, phosphoric acid, or an alpha hydroxy organic acid.

20. A cleaning and sanitizing wipe comprising a porous or absorbent sheet infused with a cleaning and antimicrobial composition, wherein the cleaning and antimicrobial composition comprises, by weight:
(a) About 0.3% of PCMX;
(b) About 2.85% of ammonium lauryl sulfate;
(c) About 2% of hexylene glycol;
(d) About 0.5% of phenoxyethanol;
(e) About 0.05% of fragrance;
(f) About 0.01% of vitamin E succinate;
(g) About 0.05% of aloe vera gel;
(h) About 0.04% of acidic calcium sulfate (“ACS”); and
(i) Remainder water.

21. A method for reducing the number of microbial organisms on a surface, comprising:
contacting the surface with the cleaning and sanitizing wipe of claim 4.

22. A method for reducing the number of Gram positive bacteria, Gram negative bacteria, or both, on a surface, comprising:
contacting the surface with the cleaning and sanitizing wipe of claim 4.

23. A method for producing a log reduction of up to about 6 in the number of Gram positive bacteria, Gram negative bacteria, or both, on a surface, comprising:
contacting the surface with the cleaning and sanitizing wipe of claim 4 for about 30 seconds or longer.
